SEQUENCE LISTING (28 sequences)

SEQ ID NO: 1 - RNA, 39 nt, synthetic construct
  source 1..39: mol_type = other RNA
gcttcgctacccgatgtattccgaaccctcstcctgcta

SEQ ID NO: 2 - DNA, 47 bp, synthetic construct
  source 1..47: mol_type = other DNA
gcccagactacacggtaatgcctgagwgtarctccctccttttctat

SEQ ID NO: 3 - DNA, 41 bp, synthetic construct
  source 1..41: mol_type = other DNA
gcttcgctacccgatgtattccstcvtcctgctrctctbgg

SEQ ID NO: 4 - DNA, 42 bp, synthetic construct
  source 1..42: mol_type = other DNA
gcccagactacacggtaatgcctgagsgyarctccctccttt

SEQ ID NO: 5 - DNA, 41 bp, synthetic construct
  source 1..41: mol_type = other DNA
gcttcgctacccgatgtattccctcmtcctgctgctctcgg

SEQ ID NO: 6 - DNA, 43 bp, synthetic construct
  source 1..43: mol_type = other DNA
gcccagactacacggtaatgycayagctccwaggacagctagg

SEQ ID NO: 7 - DNA, 40 bp, synthetic construct
  source 1..40: mol_type = other DNA
gcttcgctacccgatgtatttctgacggcgttactgatgg

SEQ ID NO: 8 - DNA, 40 bp, synthetic construct
  source 1..40: mol_type = other DNA
gcccagactacacggtaatgccagctcccgtcaatgtctt

SEQ ID NO: 9 - DNA, 40 bp, synthetic construct
  source 1..40: mol_type = other DNA
gcttcgctacccgatgtattatggtgtgtctgargytccc

SEQ ID NO: 10 - DNA, 40 bp, synthetic construct
  source 1..40: mol_type = other DNA
gcccagactacacggtaatgagctcaggaatcctsttggc

SEQ ID NO: 11 - DNA, 39 bp, synthetic construct
  source 1..39: mol_type = other DNA
gcttcgctacccgatgtattggatcccyggagrccttcg

SEQ ID NO: 12 - DNA, 42 bp, synthetic construct
  source 1..42: mol_type = other DNA
gcccagactacacggtaatggayggatrataaggccmagccc

SEQ ID NO: 13 - DNA, 56 bp, synthetic construct
  source 1..56: mol_type = other DNA
cccagtctcagtcgtgtataacgnnnnnnnnnnnnngcttcgctacccgatgtatt

SEQ ID NO: 14 - DNA, 46 bp, synthetic construct
  source 1..46: mol_type = other DNA
acatgggtggtggtatagcgctgctggcccagactacacggtaatg

SEQ ID NO: 15 - DNA, 20 bp, synthetic construct
  source 1..20: mol_type = other DNA
gcttcgctacccgatgtatt

SEQ ID NO: 16 - DNA, 20 bp, synthetic construct
  source 1..20: mol_type = other DNA
gcccagactacacggtaatg

SEQ ID NO: 17 - DNA, 19 bp, synthetic construct
  source 1..19: mol_type = other DNA
ccgaaccctcstcctgcta

SEQ ID NO: 18 - DNA, 27 bp, synthetic construct
  source 1..27: mol_type = other DNA
cctgagwgtarctccctccttttctat

SEQ ID NO: 19 - DNA, 21 bp, synthetic construct
  source 1..21: mol_type = other DNA
ccstcvtcctgctrctctbgg

SEQ ID NO: 20 - DNA, 22 bp, synthetic construct
  source 1..22: mol_type = other DNA
cctgagsgyarctccctccttt

SEQ ID NO: 21 - DNA, 21 bp, synthetic construct
  source 1..21: mol_type = other DNA
ccctcmtcctgctgctctcgg

SEQ ID NO: 22 - DNA, 23 bp, synthetic construct
  source 1..23: mol_type = other DNA
ycayagctccwaggacagctagg

SEQ ID NO: 23 - DNA, 20 bp, synthetic construct
  source 1..20: mol_type = other DNA
tctgacggcgttactgatgg

SEQ ID NO: 24 - DNA, 20 bp, synthetic construct
  source 1..20: mol_type = other DNA
ccagctcccgtcaatgtctt

SEQ ID NO: 25 - DNA, 20 bp, synthetic construct
  source 1..20: mol_type = other DNA
atggtgtgtctgargytccc

SEQ ID NO: 26 - DNA, 20 bp, synthetic construct
  source 1..20: mol_type = other DNA
agctcaggaatcctsttggc

SEQ ID NO: 27 - DNA, 19 bp, synthetic construct
  source 1..19: mol_type = other DNA
ggatcccyggagrccttcg

SEQ ID NO: 28 - DNA, 22 bp, synthetic construct
  source 1..22: mol_type = other DNA
gayggatrataaggccmagccc

(19) United States
(12) Patent Application Publication
(10) Pub. No.: US 2024/0344129 A1
(43) Pub. Date: Oct. 17, 2024

(54) COMPOSITIONS AND METHODS FOR HLA HAPLOTYPE SEQUENCING

(71) Applicants: Karen Anderson, Scottsdale, AZ (US); Oliver Kask, Chandler, AZ (US); Mark Knappenberger, Mesa, AZ (US)

(72) Inventors: Karen Anderson, Scottsdale, AZ (US); Oliver Kask, Chandler, AZ (US); Mark Knappenberger, Mesa, AZ (US)

(21) Appl. No.: 18/631,875

(22) Filed: Apr. 10, 2024

Related U.S. Application Data
(60) Provisional application No. 63/495,910, filed on Apr. 13, 2023.

(51) Int. Cl. C12Q 1/6881

(52) U.S. Cl. CPC C12Q 1/6881; C12Q 2600/16

(57) ABSTRACT

Disclosed herein are compositions and methods for performing haplotype typing. The compositions comprise nucleic acid primers for amplifying and barcoding HLA loci DNA for multiplex sequencing.

### CROSS-REFERENCE TO RELATED APPLICATIONS

This patent application claims the benefit of, and priority to, U.S. Provisional Patent Application No. 63/495,910, filed Apr. 13, 2023, the content of which is incorporated herein by reference in its entirety.

### REFERENCE TO AN ELECTRONIC SEQUENCE LISTING

The contents of the electronic sequence listing (112624.01443.xml; Size: 25,891 bytes; and Date of Creation: Apr. 8, 2024) is herein incorporated by reference in its entirety.

### BACKGROUND

Human Leukocyte Antigen (HLA) haplotype sequencing is integral to the development of personalized therapies and diagnostics, as well as to our understanding of T cell-mediated immunity. The 6 classical major HLA loci include HLA-A, HLA-B, and HLA-C(class I); and HLA-DRB1, HLA-DQB1, and HLA-DPB1 (class II), with over ˜32,000 total alleles. High-resolution typing methods currently include probe-based hybridization, nested PCR, and RNA-seq, however these protocols typically require outsourcing, take weeks of time, and are expensive for low resolution mapping, which are prohibitively expensive for large numbers of samples. Therefore, new methods to barcode HLA mRNA for downstream applications are desired.

### SUMMARY

Disclosed herein are compositions, methods, and kits useful for barcoding and sequencing HLA nucleic acids.

In an aspect, provided herein is a composition comprising one or more human leukocyte antigen (HLA) nucleic acid primer pairs, each pair comprising: a forward primer comprising: a sequence that is complementary to a cDNA sequence of an HLA locus; and a common forward primer sequence at the 5′ end of the primer; and a reverse primer comprising: a sequence that is complementary to a cDNA sequence of the HLA locus; and a common reverse primer sequence at the 5′ end of the primer, wherein each of the one or more primer pairs is complementary to a different HLA locus.

In an aspect, provided herein is a kit comprising any of the compositions described herein; nucleotides; a polymerase, and buffers necessary to carry out a RT-PCR or PCR reaction.

In an aspect, provided herein is a method of barcoding cDNA molecules from one or more HLA loci, the method comprising: (a) reverse transcribing one or more mature HLA mRNAs and amplifying the resulting HLA cDNA using one or more HLA primer pairs comprising: a forward primer comprising: a sequence that is complementary to a cDNA sequence of an HLA locus; and a common forward primer sequence at the 5′ end of the primer; and a reverse primer comprising: a sequence that is complementary to a cDNA sequence of the HLA locus; and a common reverse primer sequence at the 5′ end of the primer, wherein each of the one or more primer pairs is complementary to a different HLA locus; and (b) amplifying the cDNA from step (a) with one or more barcode primer pairs, each pair comprising: a barcode forward primer comprising the common forward primer sequence; and a barcode reverse primer comprising the common reverse primer sequence; wherein at least one of the barcode forward primer and barcode reverse primer further comprises a barcode sequence.

In an aspect, provided herein is a method of identifying an HLA allele profile of two or more mRNA samples, the method comprising; in each sample, barcoding cDNA molecules from one or more HLA loci by any of the barcoding methods described herein; wherein the one or more barcode primer pairs in each sample comprises a different barcode sequence; amplifying the barcoded cDNA molecules; pooling the samples; and sequencing the barcoded cDNA molecules in the pooled sample.

In a first aspect, provided herein is a composition comprising one or more human leukocyte antigen (HLA) primer pairs, each HLA primer pair comprising: a forward primer comprising: a sequence that is complementary to a forward strand cDNA sequence of an HLA locus; and a common forward primer sequence at the 5′ end of the forward primer; and a reverse primer comprising: a sequence that is complementary to a reverse strand cDNA sequence of the HLA locus; and a common reverse primer sequence at the 5′ end of the reverse primer, wherein each of the one or more primer pairs is complementary to a different HLA locus. In embodiments, the one or more HLA loci is selected from HLA-A, HLA-B, HLA-C, HLA-DRB1, HLA-DQB1, and HLA-DPB1.

In embodiments, the common forward primer sequence comprises SEQ ID NO: 15 and the common reverse primer sequence comprises SEQ ID NO: 16. In embodiments, the one or more HLA primer pairs comprise a forward primer sequence and a reverse primer sequence selected from: SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; and SEQ ID NO: 11 and SEQ ID NO: 12.

In embodiments, the composition further comprises a barcode primer pair, the barcode primer pair comprising: a barcode forward primer comprising the common forward primer sequence; and a barcode reverse primer comprising the common reverse primer sequence; wherein at least one of the barcode forward primer and the barcode reverse primer further comprises a barcode sequence. In embodiments, the barcode forward primer comprises the barcode sequence. In embodiments, the barcode forward primer comprises SEQ ID NO: 13 and the barcode reverse primer comprises SEQ ID NO: 14.

In another aspect, provided herein is a kit comprising the composition described herein; wherein the one or more HLA primer pairs are in a separate container than the barcode primer pairs. In embodiments, the kit comprises six HLA primer pairs; and each of the HLA primer pairs is complementary to one of an HLA-A locus, an HLA-B locus, an HLA-C locus, an HLA-DRB1 locus, an HLA-DQB1 locus, and an HLA-DPB1 locus.

In another aspect, provided herein is a method for barcoding cDNA molecules derived from one or more HLA loci, the method comprising: (a) reverse transcribing one or more mature HLA mRNAs to produce one or more template HLA cDNAs, (b) amplifying the template HLA cDNA using one or more HLA primer pairs, each HLA primer pair comprising: a forward primer comprising: a sequence that is complementary to a forward strand cDNA sequence of an HLA locus; and a common forward primer sequence at the 5′ end of the primer; and a reverse primer comprising: a sequence that is complementary to a reverse strand cDNA sequence of the HLA locus; and a common reverse primer sequence at the 5′ end of the primer; wherein each of the one or more HLA primer pairs is complementary to a different HLA locus; and wherein amplifying the template HLA cDNA produces a common HLA cDNA; and (c) amplifying the common HLA cDNA from step (b) using a barcode primer pair, the barcode primer pair comprising: a barcode forward primer comprising the common forward primer sequence; and a barcode reverse primer comprising the common reverse primer sequence; wherein at least one of the barcode forward primer and barcode reverse primer further comprises a barcode sequence; and wherein amplifying the common HLA cDNA produces a barcoded HLA cDNA.

In embodiments, the barcode forward primer comprises the barcode sequence. In embodiments, the one or more HLA loci is selected from HLA-A, HLA-B, HLA-C, HLA-DRB1, HLA-DQB1, and HLA-DPB1. In embodiments, the one or more HLA loci comprises all of HLA-A, HLA-B, HLA-C, HLA-DPB1, HLA-DRB1, and HLA-DQB1. In embodiments, the common forward primer sequence comprises SEQ ID NO: 15 and the common reverse primer sequence comprises SEQ ID NO: 16. In embodiments, the one or more HLA primer pairs comprise a forward primer sequence and a reverse primer sequence selected from: SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; and SEQ ID NO: 11 and SEQ ID NO: 12. In embodiments, the barcode forward primer comprises SEQ ID NO: 13 and the barcode reverse primer comprises SEQ ID NO: 14.

In another aspect, provided herein is a method for identifying an HLA allele profile of two or more nucleic acid samples, the method comprising: in each sample, barcoding cDNA molecules from one or more HLA loci by the methods described herein; wherein the one or more barcode primer pairs in each sample comprises a different barcode sequence; pooling the samples into a pooled sample; and sequencing the barcoded cDNA molecules in the pooled sample. In embodiments, the sequencing is performed by nanopore sequencing. In embodiments, the method further comprises preparing a cDNA library comprising the barcoded cDNA molecules. In embodiments, each sample is from a different subject.

### BRIEF DESCRIPTION OF THE DRAWINGS

FIG. 1 Workflow of RT-PCR and PCR reactions to reverse-transcribe, amplify, and barcode label HLA mRNA.

### DETAILED DESCRIPTION

Human leukocyte antigen (HLA) genes are highly polymorphic and play a critical role in guiding adaptive immune responses by presenting foreign and self peptides to T cells. There are two major classes of HLA antigens; HLA class I (HLA-A, -B and -C) and HLA class II (HLA-DR, -DQ and -DP).

HLA class I (HLA-I) and HLA class II (HLA-II) molecules present peptides that are typically recognized as a complex by CD8 and CD4 T cells, respectively. The three classical HLA-I genes expressed in all nucleated cells in humans are HLA-A, HLA-B, and HLA-C. HLA-I molecules present peptides derived from intracellular proteins. HLA-II genes (HLA-DR, HLA-DP and HLA-DQ) are constitutively expressed in only a subset of cells specialized for antigen presentation, such as dendritic cells, B cells, and macrophages, but expression can also be induced in additional cell types. HLA-II molecules present peptides derived from extracellular proteins taken into cells via endocytosis and phagocytosis, and intracellular proteins that access the HLA-II processing pathway via autophagy. The HLA system is found in humans, and is analogous to the major histocompatibility complex (MHC) system.

HLA genes are codominantly expressed and highly polymorphic, with many different alleles that modify the adaptive immune system that helps the body to distinguish its own proteins from those of foreign invaders like viruses, bacteria, and other pathogens. HLA are one of the most polymorphic genes in humans, with several thousand alleles encoding for functional polypeptides.

An HLA haplotype is a series of HLA genes inherited by a child through chromosomes, one from the mother, the other from the father. Some haplotypes are associated with autoimmune disorders and other diseases. HLA haplotype typing is used to identify HLA allele profiles, and can be used to accelerate diagnosis of such diseases. However, HLA haplotype sequencing can require extended periods of time and high costs, and can be limited to low resolution mapping. Disclosed herein are novel methods of tagging or barcoding specific HLA loci, which allows for highly multiplexed sequencing runs in which ˜100 samples can be sequenced in a single sequencing run.

### Compositions

The present disclosure provides a composition for amplifying and/or barcoding Human Leukocyte Antigen (HLA) loci. The composition comprises at least one primer pair specific for an HLA locus. Each primer pair comprises a forward primer, the forward primer comprising a sequence that is complementary to a forward strand cDNA sequence of an HLA locus, and a common forward primer sequence at the 5′ end; and a reverse primer, the reverse primer comprising a sequence that is complementary to a reverse strand cDNA sequence of the HLA locus, and a common reverse primer sequence at the 5′ end of the reverse primer. Each of the at least one primer pairs is complementary to a different HLA locus. The HLA loci may be selected from HLA-A, HLA-B, HLA-C, HLA-DPB1 HLA-DRB1, and HLA-DQB1.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

In some nucleic acid amplification methods, such as polymerase chain reaction (PCR), primers are designed to be used in pairs. A “primer pair” is a pair of primers designed to flank a target DNA region of interest, one primer hybridizing to the forward strand, the other primer hybridizing to the reverse strand.

In exemplary embodiments the primer pairs of the composition disclosed herein comprise or consist of sequences having at least 90%, 91%, 92%, 93%, 94%, 95%, 96%, 97%, 98%, 99%, or 100% identity to the sequences of: SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; and SEQ ID NO: 11 and SEQ ID NO: 12, which bind to the HLA-A, HLA-B, HLA-C, HLA-DPB1 HLA-DRB1, and HLA-DQB1 loci, respectively (see Table 1). In embodiments, the segments of the forward and reverse primers that hybridize to the cDNA comprise 100% complementarity to the cDNA. For example, the sequence complementary to a forward strand cDNA sequence of an HLA locus may comprise or consist of SEQ ID NO: 17, 19, 21, 23, 25, Or 27, which bind to the HLA-A, HLA-B, HLA-C, HLA-DPB1 HLA-DRB1, and HLA-DQB1 loci, respectively; and the reverse strand cDNA sequence of the HLA locus may comprise or consist of SEQ ID NO: 18, 20, 22, 24, 26, or 28, which bind to the HLA-A, HLA-B, HLA-C, HLA-DPB1 HLA-DRB1, and HLA-DQB1 loci, respectively.

The terms “common forward primer sequence” and “common reverse primer sequence”, collectively “the common sequences”, as used herein, refer to segments of the forward and reverse primers that are not complementary to and do not hybridize with the target cDNA sequence, e.g. the HLA cDNA. The common sequences incorporate additional features which allow for the detection and/or immobilization of the primers but do not alter the basic property of the primers, that of acting as a point of initiation of DNA synthesis. The common sequences may facilitate cloning or detection of the amplified product, or enable transcription of RNA or translation of protein (for example, by inclusion of a 5′-UTR, such as an Internal Ribosome Entry Site (IRES) or a 3′-UTR element, such as a poly (A)ₙ sequence, where n is in the range from about 20 to about 200). The region of the primer that is sufficiently complementary to the template to hybridize is referred to herein as the hybridizing region. In exemplary embodiments, the common forward primer sequence and common reverse primer sequence have at least 90%, 91%, 92%, 93%, 94%, 95%, 96%, 97%, 98%, 99%, or 100% identity to the sequences of SEQ ID NO: 15 and SEQ ID NO: 16, respectively.

The terms “target, “target sequence”, “target region”, and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular CloningA Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

The composition may further comprise one or more barcode primer pairs, each barcode primer pair comprising a barcode forward primer comprising the common forward primer sequence (e.g. SEQ ID NO: 15); and a barcode reverse primer comprising the common reverse primer sequence (e.g. SEQ ID NO: 16), wherein at least one of the barcode forward primer and barcode reverse primer further comprises a barcode sequence. The barcode primers are able to hybridize to a sequence complementary to the common sequences, which are incorporated into cDNA molecules that are produced after amplification by the HLA primers. In exemplary embodiments, the barcode sequence is on the barcode forward primer. The barcode forward primer and barcode reverse primer may also comprise a buffer sequence. By way of example, but not limitation a barcode forward primer may comprise a sequence having at least 90%, 91%, 92%, 93%, 94%, 95%, 96%, 97%, 98%, 99%, or 100% identity to SEQ ID NO: 13, wherein the Xs represent a barcode sequence. In exemplary embodiments, the barcode reverse primer comprises a sequence having at least 90%, 91%, 92%, 93%, 94%, 95%, 96%, 97%, 98%, 99%, or 100% identity to SEQ ID NO: 14. Preferably, the segments of the barcode forward and barcode reverse primers that hybridize to the cDNA (i.e. the common forward primer sequence and the common reverse primer sequence) comprise 100% complementarity to the cDNA being barcoded.

The terms “tag” and “barcode,” are used interchangeably herein, and generally refer to a nucleic acid sequence that may be attached to a nucleic acid analyte to convey information about the analyte. For example, a barcode may be a polynucleotide sequence attached to fragments of a target polynucleotide contained within a particular partition. This barcode may then be sequenced with the fragments of the target polynucleotide. The presence of the same barcode on multiple sequences may provide information about the origin of the sequences. For example, a barcode may indicate that the sequence came from a particular subject or sample. This is particularly useful for sequence assembly when several samples are pooled before sequencing. As such, the barcode sequence is an identifiable sequence that serves as a tag for identifying the source of the amplified cDNA molecule.

The terms “nucleic acid,” “nucleic acid molecule,” “oligonucleotide,” and “polynucleotide” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include double- and single-stranded deoxyribonucleic acids (DNA) (including genomic DNA and cDNA), and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Methods of making polynucleotides of a predetermined sequence are well-known. Sec, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. Sec, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine. In the context of the present disclosure abbreviation for common nucleotides and nucleotide alternatives are used. “M” refers to adenosine or cytosine, “R” refers to adenosine or guanosine, “W” refers to adenosine or thymidine/uridine, “S” refers to cytosine or guanosine, “Y” refers to cytosine or thymidine/uridine, “K” refers to guanosine or thymidine/uridine, “V” refers to adenosine or cytosine or guanosine, “H” refers to adenosine or cytosine or thymidine/uridine, “D” refers to adenosine or guanosine or thymidine/uridine, “B” refers to cytosine or guanosine or thymidine/uridine, “N” and “X” refers to adenosine or cytosine or guanosine or thymidine/uridine.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. In embodiments of the present disclosure primers are complementary to the HLA target. For example, the primers may be complementary to nucleotides within HLA-A, HLA-B, HLA-C, HLADPB, HLA-DRB1, and HLA-DQB1.

“Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be evaluated using the Basic Local Alignment Search Tool (“BLAST”), which is well known in the art (Karlin and Altschul, Methods for assessing the statistical significance of molecular sequence features by using general scoring schemes. Proc. Natl. Acad. Sci. USA (1990) 87:2267-2268; Altschul et al. Gapped BLAST and PSI-BLAST: a new generation of protein database search programs. Nucl. Acids Res. (1997) 25:3389-3402). The BLAST programs identify homologous sequences by identifying similar segments between a query amino acid or nucleic acid sequence and a test sequence, which is preferably obtained from a protein or nucleic acid sequence database. The BLAST programs can be used with the default parameters or with modified parameters provided by the user.

### Kits

The present disclosure also provides a kit for preparing barcoded HLA cDNAs comprising the HLA primer pairs and barcode primer pairs described herein, wherein the HLA primer pairs and barcode primer pairs are provided in separate packaging/containers. The HLA primer pairs in the kit may comprise those of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; and SEQ ID NO: 11 and SEQ ID NO: 12, as described herein. The kit may comprise all of the described HLA primer pairs. The kit may further comprise reagents for carrying out a DNA amplification reaction. For example, the kit may comprise reagents and buffers necessary for PCR and RT-PCR.

### Methods:

The present disclosure also provides a method for barcoding a cDNA molecule derived from an HLA locus. The method comprises (a) reverse transcribing a mature HLA mRNA to produce a template HLA cDNA; (b) amplifying the template HLA cDNA using one or more HLA primer pairs, each HLA primer pair comprising a forward primer that comprises a sequence that is complementary to a forward strand cDNA sequence of an HLA locus; and a common forward primer sequence at the 5′ end of the primer; and a reverse primer that comprises a sequence that is complementary to a reverse strand cDNA sequence of the HLA locus, and a common reverse primer sequence at the 5′ end of the primer; and (c) amplifying the common HLA cDNA from step (b) using a barcode primer pair, the barcode primer pair comprising a barcode forward primer comprising the common forward primer sequence; and a barcode reverse primer comprising the common reverse primer sequence; wherein at least one of the barcode forward primer and barcode reverse primer further comprises a barcode sequence; and wherein amplifying the common HLA cDNA produces a barcoded HLA cDNA. In exemplary embodiments, the barcode sequence is on the barcode forward primer. The method may comprise barcoding additional cDNA molecules, each from a different HLA locus. The HLA locus may be selected from HLA-A, HLA-B, HLA-C, HLA-DRB1, HLA-DQB1, and HLA-DPB1. The method may comprise barcoding cDNA derived from each of HLA-A, HLA-B, HLA-C, HLA-DRB1, HLA-DQB1, and HLA-DPB1.

The HLA primer pairs may comprise any of the HLA primer pairs described herein. The HLA primer pair sequences may comprise or consist of sequences having at least 90%, 91%, 92%, 93%, 94%, 95%, 96%, 97%, 98%, 99%, or 100% identity to the sequences of: SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; and SEQ ID NO: 11 and SEQ ID NO: 12. The common forward primer sequence and common reverse primer sequence may have at least 90%, 91%, 92%, 93%, 94%, 95%, 96%, 97%, 98%, 99%, or 100% identity to the sequences of SEQ ID NO: 15 and SEQ ID NO:16, respectively. The barcode forward primer may comprise a sequence having at least 90%, 91%, 92%, 93%, 94%, 95%, 96%, 97%, 98%, 99%, or 100% identity to SEQ ID NO: 13, wherein the Xs represent a barcode sequence. The barcode reverse primer may comprise a sequence having at least 90%, 91%, 92%, 93%, 94%, 95%, 96%, 97%, 98%, 99%, or 100% identity to SEQ ID NO: 14.

The synthesis of DNA from an RNA template, via reverse transcription, produces complementary DNA (cDNA). Reverse transcriptases (RTs) use an RNA template and a short primer complementary to the 3′ end of the RNA to direct the synthesis of the first strand cDNA, which can be used directly as a template for the Polymerase Chain Reaction (PCR). Alternatively, a first-strand cDNA can be made double-stranded using DNA Polymerase I and DNA Ligasc.

The term “amplifying” or “copying” as used herein generally refers to the production of a plurality of nucleic acid molecules from a target nucleic acid wherein primers hybridize to specific sites on the target nucleic acid molecules in order to provide an initiation site for extension by a polymerase. Amplification can be carried out by any method generally known in the art, such as but not limited to: standard PCR, long PCR, hot start PCR, qPCR, RT-PCR and Isothermal Amplification. Other amplification reactions comprise, among others, the Ligase Chain Reaction, Polymerase Ligase Chain Reaction, Gap-LCR, Repair Chain Reaction, 3 SR, NASBA, Strand Displacement Amplification (SDA), Transcription Mediated Amplification (TMA), and Q3-amplification.

The term “amplification reaction” refers to any chemical reaction, including an enzymatic reaction, which results in increased copies of a template nucleic acid sequence or results in transcription of a template nucleic acid. For example, an amplification reaction may include reverse transcription and the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcusfuriosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” and “transcription template” refer to a nucleic acid that serves as substrate for transcribing at least one RNA that can be translated into a polypeptide or protein. Expression templates include nucleic acids composed of DNA or RNA Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

As used herein, “translation template” refers to an RNA product of transcription from an expression template that can be used by ribosomes to synthesize polypeptide or protein.

The term “reaction mixture,” as used herein, refers to a solution containing reagents necessary to carry out a given reaction. A reaction mixture is referred to as complete if it contains all reagents necessary to enable the reaction, and incomplete if it contains only a subset of the necessary reagents.

An “amplification reaction mixture”, which refers to a solution containing reagents necessary to carry out an amplification reaction, typically contains oligonucleotide primers and a DNA polymerase in a suitable buffer.

A “PCR reaction mixture”, which refers to a solution containing the reagents necessary to carry out a PCR reaction, typically contains DNA polymerase, dNTPs, and a divalent metal cation in a suitable buffer.

The present disclosure also provides a method for identifying an HLA allele profile of two or more nucleic acid samples. The method comprises, in each sample, barcoding cDNA molecules from one or more HLA loci using any of the barcoding methods described herein, wherein the one or more barcode primer pairs in each sample comprises a different barcode sequence; pooling the samples into a pooled sample; and sequencing the barcoded cDNA molecules in the pooled sample. In embodiments, each of the two or more samples are from a different subject.

“Sequencing” is the process of determining the order of nucleotides in a polynucleotide. Any suitable sequencing technique may be used with the present methods, including next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), or paired-end sequencing. In exemplary embodiments, nanopore sequencing is used.

The nucleic acid sample may be extracted from a clinical sample obtained from a subject. Appropriate clinical samples may include a biopsy, scraping, swab, blood, mucus, urine, stool, plasma, semen, hair, etc. The “subject” from which the sample is derived may be a mammal. In preferred embodiments, the subject is a human.

The present disclosure also provides a method for synthesizing or preparing a library of HLA polynucleotides comprising barcode sequences, the library comprising cDNAs prepared by any of the methods disclosed herein.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following examples are meant only to be illustrative and are not meant as limitations on the scope of the invention or of the appended claims.

### Example 1

In the following example, the inventors describe methods to reverse-transcribe, amplify, and barcode label HLA mRNA.

Disclosed herein are novel methods of barcoding human HLA mRNA for downstream use. The method comprises two steps: 1. RT-PCR to specifically reverse transcribe and amplify the mRNA of six HLA loci to cDNA, and 2. PCR to amplify and barcode cDNA of all six HLA loci.

1. Reverse Transcription-Polymerase Chain Reaction (RT-PCR) primer sets for specifically reverse transcribing and amplifying the mRNA of six HLA loci to cDNA

Six primer pairs have been designed that are complementary to sequences found at the ends of mature mRNA for six HLA loci. Specifically, these loci are HLA-A, HLA-B, HLA-C, HLA-DPB1 HLA-DRB1, and HLA-DQB1. The primers are utilized in a multiplex RT-PCR reaction where all six sets are combined to produce amplified cDNA. All 12 primers have a common sequence attached to the 5′ ends which is used for further amplification with barcoded primer sets.

TABLE 1
List of 12 Loci Specific Primers, sequences complimentary to cDNA
are in bold.

| Primer Name | Primer Sequence | SEQ ID NO: |
|---|---|---|
| HLA-A- | GCTTCGCTACCCGATGTATTCCGAACCCTCSTCCT | 1 |
| Forward | GCTA | |
| HLA-A- | GCCCAGACTACACGGTAATGCCTGAGWGTARCTC | 2 |
| Reverse | CCTCCTTTTCTAT | |
| HLA-B- | GCTTCGCTACCCGATGTATTCCSTCVTCCTGCTRCT | 3 |
| Forward | CTBGG | |
| HLA-B- | GCCCAGACTACACGGTAATGCCTGAGSGYARCTCC | 4 |
| Reverse | CTCCTTT | |
| HLA-C- | GCTTCGCTACCCGATGTATTCCCTCMTCCTGCTGC | 5 |
| Forward | TCTCGG | |
| HLA-C- | GCCCAGACTACACGGTAATGYCAYAGCTCCWAGG | 6 |
| Reverse | ACAGCTAGG | |
| HLA-DPB1- | GCTTCGCTACCCGATGTATTTCTGACGGCGTTACT | 7 |
| Forward | GATGG | |
| HLA-DPB1- | GCCCAGACTACACGGTAATGCCAGCTCCCGTCAAT | 8 |
| Reverse | GTCTT | |
| HLA-DRB1- | GCTTCGCTACCCGATGTATTATGGTGTGTCTGARG | 9 |
| Forward | YTCCC | |
| HLA-DRB1- | GCCCAGACTACACGGTAATGAGCTCAGGAATCCTS | 10 |
| Reverse | TTGGC | |
| HLA-DQB1- | GCTTCGCTACCCGATGTATTGGATCCCYGGAGRCC | 11 |
| Forward | TTCG | |
| HLA-DQB1- | GCCCAGACTACACGGTAATGGAYGGATRATAAGG | 12 |
| Reverse | CCMAGCCC | |

2. Polymerase Chain Reaction primer set which amplifies cDNA of all six HLA loci and include 13-nucleotide long DNA barcodes used for mapping cDNA molecules to specific samples

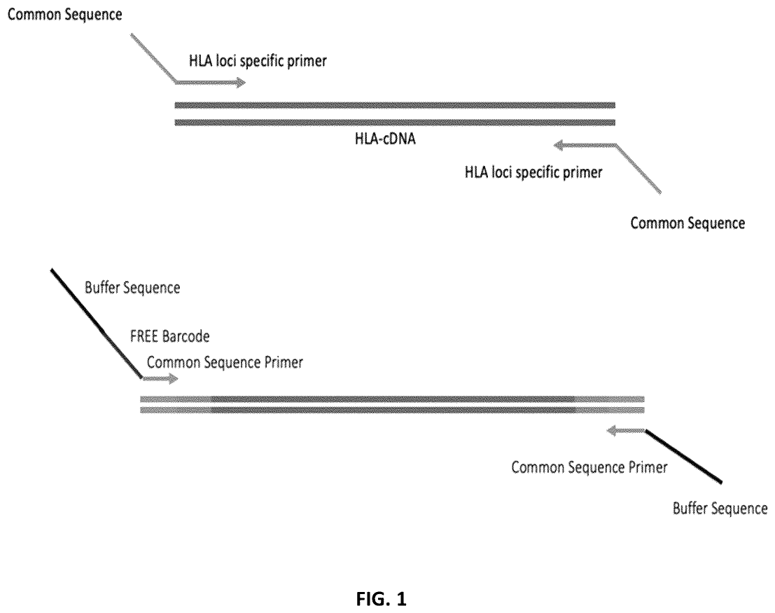

A primer pair has been designed to further amplify all six loci cDNA from the RT-PCR reaction. This primer pair utilizes the common sequences on the 5′ ends of the primers presented in step 1. Attached to the 5′ end of the forward primer is a 13-nucleotide long DNA barcode used to identify which sample each cDNA came from. The products of the PCR using this primer pair are used for long read nanopore sequencing. The HLA locus amplification scheme is depicted in FIG. 1.

TABLE 2
List of primers and their sequences used in the 2^{nd} step PCR reaction. Xs
denote the nucleotides which make up the barcode sequence.

| Primer Name | Primer Sequence | SEQ ID NO: |
|---|---|---|
| Barcoded-PhII- | CCCAGTCTCAGTCGTGTATAACGXXXXXXXX | 13 |
| Forward | XXXXXGCTTCGCTACCCGATGTATT | |
| PhII-Reverse | ACATGGGTGGTGGTATAGCGCTGCTGGCCCA | 14 |
| | GACTACACGGTAATG | |

### CONCLUSIONS

These disclosures represent novel advancements for the reverse transcription, amplification, and barcode labeling of HLA mRNA for downstream long read nanopore sequencing.

SEQUENCES

| Description | Sequence | SEQ ID NO: |
|---|---|---|
| HLA-A-Forward Primer | GCTTCGCTACCCGATGTATTCCGAACCCT | 1 |
| | CSTCCTGCTA | |
| HLA-A-Reverse Primer | GCCCAGACTACACGGTAATGCCTGAGWG | 2 |
| | TARCTCCCTCCTTTTCTAT | |
| HLA-B-Forward Primer | GCTTCGCTACCCGATGTATTCCSTCVTCC | 3 |
| | TGCTRCTCTBGG | |
| HLA-B-Reverse Primer | GCCCAGACTACACGGTAATGCCTGAGSG | 4 |
| | YARCTCCCTCCTTT | |
| HLA-C-Forward Primer | GCTTCGCTACCCGATGTATTCCCTCMTCC | 5 |
| | TGCTGCTCTCGG | |
| HLA-C-Reverse Primer | GCCCAGACTACACGGTAATGYCAYAGCT | 6 |
| | CCWAGGACAGCTAGG | |
| HLA-DPB1-Forward | GCTTCGCTACCCGATGTATTTCTGACGGC | 7 |
| Primer | GTTACTGATGG | |
| HLA-DPB1-Reverse | GCCCAGACTACACGGTAATGCCAGCTCC | 8 |
| Primer | CGTCAATGTCTT | |
| HLA-DRB1-Forward | GCTTCGCTACCCGATGTATTATGGTGTGT | 9 |
| Primer | CTGARGYTCCC | |
| HLA-DRB1-Reverse | GCCCAGACTACACGGTAATGAGCTCAGG | 10 |
| Primer | AATCCTSTTGGC | |
| HLA-DQB1-Forward | GCTTCGCTACCCGATGTATTGGATCCCYG | 11 |
| Primer | GAGRCCTTCG | |
| HLA-DQB1-Reverse | GCCCAGACTACACGGTAATGGAYGGATR | 12 |
| Primer | ATAAGGCCMAGCCC | |
| Barcoded-PhII-Forward | CCCAGTCTCAGTCGTGTATAACGXXXXXX | 13 |
| Primer | XXXXXXXGCTTCGCTACCCGATGTATT | |
| PhII-Reverse Primer | ACATGGGTGGTGGTATAGCGCTGCTGGCC | 14 |
| | CAGACTACACGGTAATG | |
| Forward Primer Common | GCTTCGCTACCCGATGTATT | 15 |
| Sequence | | |
| Reverse Primer Common | GCCCAGACTACACGGTAATG | 16 |
| Sequence | | |
| HLA-A cDNA forward | CCGAACCCTCSTCCTGCTA | 17 |
| complementary | | |
| HLA-A cDNA reverse | CCTGAGWGTARCTCCCTCCTTTTCTAT | 18 |
| complementary | | |
| HLA-B cDNA forward | CCSTCVTCCTGCTRCTCTBGG | 19 |
| complementary | | |
| HLA-B cDNA reverse | CCTGAGSGYARCTCCCTCCTTT | 20 |
| complementary | | |
| HLA-C cDNA forward | CCCTCMTCCTGCTGCTCTCGG | 21 |
| complementary | | |
| HLA-C cDNA reverse | YCAYAGCTCCWAGGACAGCTAGG | 22 |
| complementary | | |
| HLA-DPB1 cDNA | TCTGACGGCGTTACTGATGG | 23 |
| forward complementary | | |
| HLA-DPB1 cDNA | CCAGCTCCCGTCAATGTCTT | 24 |
| reverse complementary | | |
| HLA-DRB1 cDNA | ATGGTGTGTCTGARGYTCCC | 25 |
| forward complementary | | |
| HLA-DRB1 cDNA | AGCTCAGGAATCCTSTTGGC | 26 |
| reverse complementary | | |
| HLA-DQB1 cDNA | GGATCCCYGGAGRCCTTCG | 27 |
| forward complementary | | |
| HLA-DQB1 cDNA | GAYGGATRATAAGGCCMAGCCC | 28 |
| reverse complementary | | |

What is claimed:

1. A composition comprising one or more human leukocyte antigen (HLA) primer pairs, each HLA primer pair comprising:
a forward primer comprising:
a sequence that is complementary to a forward strand cDNA sequence of an HLA locus; and
a common forward primer sequence at the 5′ end of the forward primer; and
a reverse primer comprising:
a sequence that is complementary to a reverse strand cDNA sequence of the HLA locus; and
a common reverse primer sequence at the 5′ end of the reverse primer,
wherein each of the one or more primer pairs is complementary to a different HLA locus.

2. The composition of any of the previous claims wherein the one or more HLA loci is selected from HLA-A, HLA-B, HLA-C, HLA-DRB1, HLA-DQB1, and HLA-DPB1.

3. The composition of claim 1, wherein the common forward primer sequence comprises SEQ ID NO: 15 and the common reverse primer sequence comprises SEQ ID NO: 16.

4. The composition of claim 1, wherein the one or more HLA primer pairs comprise a forward primer sequence and a reverse primer sequence selected from:
SEQ ID NO: 1 and SEQ ID NO: 2;
SEQ ID NO: 3 and SEQ ID NO: 4;
SEQ ID NO: 5 and SEQ ID NO: 6;
SEQ ID NO: 7 and SEQ ID NO: 8;
SEQ ID NO: 9 and SEQ ID NO: 10; and
SEQ ID NO: 11 and SEQ ID NO: 12.

5. The composition of claim 1, further comprising a barcode primer pair, the barcode primer pair comprising:
a barcode forward primer comprising the common forward primer sequence; and
a barcode reverse primer comprising the common reverse primer sequence;
wherein at least one of the barcode forward primer and the barcode reverse primer further comprises a barcode sequence.

6. The composition of claim 5, wherein the barcode forward primer comprises the barcode sequence.

7. The composition of claim 5, wherein the barcode forward primer comprises SEQ ID NO: 13 and the barcode reverse primer comprises SEQ ID NO: 14.

8. A kit comprising the composition of claim 5; wherein the one or more HLA primer pairs are in a separate container than the barcode primer pairs.

9. The kit of claim 8, wherein the kit comprises six HLA primer pairs; and wherein each of the HLA primer pairs is complementary to one of an HLA-A locus, an HLA-B locus, an HLA-C locus, an HLA-DRB1 locus, an HLA-DQB1 locus, and an HLA-DPB1 locus.

10. A method for barcoding cDNA molecules derived from one or more HLA loci, the method comprising:
(a) reverse transcribing one or more mature HLA mRNAs to produce one or more template HLA cDNAs,
(b) amplifying the template HLA cDNA using one or more HLA primer pairs, each HLA primer pair comprising:
a forward primer comprising:
a sequence that is complementary to a forward strand cDNA sequence of an HLA locus; and
a common forward primer sequence at the 5′ end of the primer; and
a reverse primer comprising:
a sequence that is complementary to a reverse strand cDNA sequence of the HLA locus; and
a common reverse primer sequence at the 5′ end of the primer;
wherein each of the one or more HLA primer pairs is complementary to a different HLA locus; and
wherein amplifying the template HLA cDNA produces a common HLA cDNA; and
(c) amplifying the common HLA cDNA from step (b) using a barcode primer pair, the barcode primer pair comprising:
a barcode forward primer comprising the common forward primer sequence; and
a barcode reverse primer comprising the common reverse primer sequence;
wherein at least one of the barcode forward primer and barcode reverse primer further comprises a barcode sequence; and
wherein amplifying the common HLA cDNA produces a barcoded HLA cDNA.

11. The method of claim 10, wherein the barcode forward primer comprises the barcode sequence.

12. The method of claim 10, wherein the one or more HLA loci is selected from HLA-A, HLA-B, HLA-C, HLA-DRB1, HLA-DQB1, and HLA-DPB1.

13. The method of claim 10, wherein the one or more HLA loci comprises all of HLA-A, HLA-B, HLA-C, HLA-DPB1, HLA-DRB1, and HLA-DQB1.

14. The method of claim 10, wherein the common forward primer sequence comprises SEQ ID NO: 15 and the common reverse primer sequence comprises SEQ ID NO: 16.

15. The method of claim 10, wherein the one or more HLA primer pairs comprise a forward primer sequence and a reverse primer sequence selected from:
SEQ ID NO: 1 and SEQ ID NO: 2;
SEQ ID NO: 3 and SEQ ID NO: 4;
SEQ ID NO: 5 and SEQ ID NO: 6;
SEQ ID NO: 7 and SEQ ID NO: 8;
SEQ ID NO: 9 and SEQ ID NO: 10; and
SEQ ID NO: 11 and SEQ ID NO: 12.

16. The method of claim 10, wherein the barcode forward primer comprises SEQ ID NO: 13 and the barcode reverse primer comprises SEQ ID NO: 14.

17. A method for identifying an HLA allele profile of two or more nucleic acid samples, the method comprising:
in each sample, barcoding cDNA molecules from one or more HLA loci by the method of claim 10;
wherein the one or more barcode primer pairs in each sample comprises a different barcode sequence;
pooling the samples into a pooled sample; and
sequencing the barcoded cDNA molecules in the pooled sample.

18. The method of claim 17, wherein the sequencing is performed by nanopore sequencing.

19. The method of claim 17, further comprising preparing a cDNA library comprising the barcoded cDNA molecules.

20. The method of claim 17, wherein each sample is from a different subject.
